(19) United States
(12) Patent Application Publication
(10) Pub. No.: US 2007/0178526 A1
(43) Pub. Date: Aug. 2, 2007

(54) Use of protein profiles in disease diagnosis and treatment

(76) Inventors: Stilianos E. Kountakis, Martinez, GA (US); Bao-Ling Adam, Martinez, GA (US)

Correspondence Address: KILPATRICK STOCKTON LLP - M0351, 1001 WEST FOURTH STREET, WINSTON-SALEM, NC, 27101, US

(21) Appl. No.: 11/490,001

(22) Filed: Jul. 20, 2006

Related U.S. Application Data
(60) Provisional application No. 60/700,831, filed on Jul. 20, 2005.

(51) Int. Cl. G01N 33/53; G06F 19/00

(52) U.S. Cl. 435/7.1; 702/19

(57) ABSTRACT

Disclosed are methods, systems, and articles of manufacture for using proteomics for the diagnosis and treatment of diseases. The methods may be used to diagnose a particular disease or disease subtype. For example, the methods may be used to diagnose chronic rhinosinusitis and diseases related thereto.

### RELATED APPLICATIONS

This application claims priority to U.S. Provisional Patent Application No. 60/700,831, filed Jul. 20, 2005. The disclosure of U.S. Provisional Patent Application No. 60/700,831 is incorporated by reference in its entirety.

### FIELD OF INVENTION

The present invention relates to the use of protein profiles in disease diagnosis and treatment.

### BACKGROUND

Many common diseases are difficult to diagnose with certainty, thereby potentially compromising treatment. For example, chronic rhinosinusitis (CRS) is a common disorder, affecting approximately 10-15% of the population in the United States and costing over 3 billion dollars per year (NIH Data Book, 1990, Bethesda, Md.; US Department of Health and Human Services, 1990 Publication 90-1261; Kaliner et al., J. Allergy Clin. Immunol., 1997, 99:S829-48). The definition of CRS is based on non-disease specific symptoms, which can result from a variety of sinonasal and neurologic disorders (Kaliner et al., 1997; Lanza et al., Otolaryngol. Head Neck Surg., 1997, 117:51-7). Since the working definition of CRS does not account for the different disease subtypes, each of which may have different degrees of severity and prognosis, a discrepancy between objective measures and patient symptoms or surgical outcomes may occur. Therefore, outcome studies involving patients with “chronic rhinosinusitis” may be inaccurate unless they acknowledge the different disease subtypes within this chronic disorder. This may explain why proposed radiographic and clinical staging systems may have low specificity when they are correlated with patient symptoms and outcomes. For example, in some cases there is no association between computed tomography (CT) staging and symptom scores in patients with CRS (Stewart et al., Am. J. Rhinol., 1999, 13:161-7). In addition, it has been reported that symptom improvement may not correlate with objective improvement as assessed by nasal endoscopy (Vieming et al., Am. J. Rhinol., 1990, 4:13-7).

One of the CRS subtypes is eosinophilic chronic hyperplastic rhinosinusitis (ECHRS). While ECHRS may have many similarities to infectious or noneosinophilic obstructive/inflammatory rhinosinusitis, the exact relationship between these disease subtypes is not clear. Eosinophilic chronic hyperplastic rhinosinusitis is generally characterized by significant inflammation and thickening of the sinus mucosa, usually leading to the formation of polyps and hence, the name hyperplastic rhinosinusitis. Histologically, ECHRS may be manifested by accumulation of eosinophils, activated mast cells, fibroblasts and goblet cells (Kaliner et al., 1997; Hamilos et al., J. Allergy Clin. Immunol., 1995,96:537-44). ECHRS may frequently be associated with the presence of asthma, thus implying that the two may have a shared pathophysiology. As with asthma, the presence of activated eosinophils in the sinus tissue is the histological hallmark of ECHRS. Along with activated Th2 lymphocytes, the activated eosinophils found in hyperplastic sinus tissue (polyps) have been shown to produce a number of inflammatory cytokines including GM-CSF, IL-3, IL-4, and IL-5 (Hamilos et al., 1995; Hamilos, J. Allergy Clin. Immunol., 1993, 92:39-48; Finotto et al., J. Immunol., 1994, 153:2278-89). These cytokines are potent inflammatory mediators and are thought to contribute to the significant mucosal inflammation seen in this disease. In addition to cytokines, the molecular inflammatory proteins or substances found in this disease include cysteinyl leukotrienes (CysLT) which are products of arachidonic acid metabolism via the 5-lipooxygenase pathway (Sanak et al., Clin. Exp. Allergy, 1999, 29:306-13). It has also been shown that CysLT levels may be elevated in asthmatic patients undergoing Endoscopic Sinus Surgery (ESS) for CRS compared to patients without asthma (Arango et al., Laryngoscope, 2002, 112:1190-2). Others also described elevated CysLT levels in patients with hyperplastic sinusitis and nasal polyposis compared to patients without polyps (Steinke et al., J. Allergy Clin. Immunol., 2003, 111:342-9). Chronic rhinosinusitis generally manifests an inflammatory component regardless of the cause of the disease. Expression of inflammatory cells and molecules may vary between patients and this may contribute to the difficulty of establishing a repeating and verifiable classification that may help assign severity of disease and clinical outcomes. Disease severity may be determined based on symptom scores, sinus CT grade, endoscopy scores and findings, and surgical outcomes.

Previously, investigators would evaluate clinical objective and subjective parameters in conjunction with molecular, cellular and histologic markers in patients undergoing surgery for CRS, in order to include the various mucosal inflammatory processes present in a severity classification system for CRS. The analysis often indicated that disease severity may correlate with the presence or absence of polyps (clinical objective parameter) and the presence or absence of sinus tissue eosinophilia (a histologic marker). All other parameters did not appear to significantly contribute to this correlation with disease severity. For example, it is accepted that asthmatics may have worse sinus disease than nonasthmatics, but the patients with asthma are more likely to have polyps, which can thereby serve as a surrogate marker for asthma (Kountakis et al., Laryngoscope, 2004, 114:1895-1905). This type of classification system may help in understanding the severity of inflammation a patient is experiencing and what the possible treatment outcomes will be, but does not give information on the specific inflammatory profile of the disease. In addition, this classification system generally requires pathologic examination of sinus tissue and thus, is not readily adaptable in the office setting.

Thus, what is needed is a way to correlate molecular markers with the disease subtype, so as to reliably diagnose the disease, and to determine a prognosis regarding the severity of the disease, so as to provide the most effective methods of treatment.

### SUMMARY

The present invention relates to the use of protein profiles in disease diagnosis and treatment. The present invention may be embodied in a variety of ways.

In an embodiment, the present invention comprises a method for identifying a disease in a subject. The method may comprise the step of generating a protein profile comprising at least one protein from a sample isolated from the subject. The method may also comprise the step of comparing the protein profile for the subject to a reference protein profile. In additional embodiments, the method may also comprise the step of correlating the protein profile with a disease of interest. The method may be used for the identification of almost any disease. In an embodiment, the disease is chronic rhinosinusitis or a subtype of chronic rhinosinusitis

In other embodiments, the present invention may comprises a method for identifying a protein that is expressed as a result of a developing a particular disease. The method for identification may comprise the step of isolating samples from a plurality of subjects that exhibit the symptoms of the disease of interest. Also, the method may comprise generating a protein profile for each of the samples. The method may additionally comprise correlating expression of at least one individual protein identified in at least some of the protein profiles to the disease of interest.

In other embodiments, the present invention may provide a system for determining a diagnosis and/or prognosis of a specific disease in a subject. In an embodiment, the system may comprise a component for generating a protein profile comprising at least one protein from a sample isolated from a subject. The system may also comprise a component for comparing the protein profile for the subject to a reference protein profile. In some embodiments, the system may also comprise a component for correlating the protein profile with a disease of interest.

Other embodiments of the present invention may comprise articles of manufacture. For example, in a embodiment, the present invention may comprise an article of manufacture comprising a protein profile captured on a medium, wherein the protein profile comprises at least one protein that is correlated with the development and/or propagation of a disease of interest in a subject.

In certain embodiments, the present invention may provide certain advantages. For example, the methods, systems, or articles of manufacture of the present invention allow for the detection of multiple diagnostic and/or prognostic markers for complex diseases. Such markers may include multiple disease-related proteins, as well as other biomolecules, such as cytokines, hormones, and modulator compounds, antibodies, white blood cells or other immunological agents.

Or, the methods, systems or articles of manufacture of the present invention allow for the diagnosis of particular disease subtypes. For example, the methods, systems, or articles of manufacture of the present invention may be used to diagnose chronic rhinosinusitis (CRS) or subtypes of CRS.

In yet other embodiments, the methods, systems, or articles of manufacture of the present invention enable isolation of biomolecules, such as polypeptides or other agents, that may be used as therapeutic agents.

As another potential advantage, use of methods, systems, or articles of manufacture of the present invention for the analysis of protein profiles may be faster than other protein analysis techniques, such as 2D gel electrophoresis. Also, in certain embodiments, the methods, systems, or articles of manufacture provided by the present invention may comprise a high throughput capability. As yet another potential advantage, methods, systems, or articles of manufacture of the present invention may require orders of magnitude lower amounts of the protein sample than is required for the analysis of protein profiles by other protein analysis techniques. Also, using the methods, systems, or articles of manufacture of the present invention may allow for the effective resolution of low mass proteins, e.g., 2-20 kiloDaltons (kDa). As another advantage, the methods, systems, or articles of manufacture of the present invention may be directly applicable for clinical assay development.

There are additional features of the invention which will be described hereinafter. It is to be understood that the invention is not limited in its application to the details set forth in the following claims, description and figures. The invention is capable of other embodiments and of being practiced or carried out in various ways.

### BRIEF DESCRIPTION OF THE FIGURES

FIG. 1 shows the generation and use of a protein profile for at least one of identification, prognosis, and/or treatment of a disease, as well as for the purification of a protein related to the disease in accordance with certain embodiments of the present invention.

FIG. 2 shows a method for preparing a diagnostic protein profile in accordance with an embodiment of the present invention.

FIG. 3 shows a diagram of Biomarker Pattern Analysis utilized in the classification of chronic sinusitis (CS) (solid) and healthy, negatively screened controls (open) in accordance with an embodiment of the present invention. Each node represents an analytical splitting rule where the samples are split into two daughter nodes. Each node displays the peak mass, the cutoff intensity level, the number of samples and the composition of the samples. Terminal nodes are classified as either Control or CRS.

FIG. 4 shows a representative spectra of chronic sinusitis (CRS) samples to illustrate the first splitting rule in the classification tree of CRS and controls (CON) in accordance with an embodiment of the present invention. The peak of 8013.54 Da (tick mark on peak) appears to be slightly upregulated in CRS patients compared to healthy negatively screened controls (CON).

FIG. 5 shows a method for purification of a protein identified as being correlated with a disease of interest in accordance with an embodiment of the present invention.

FIG. 6 shows a system for generating and using protein profiles for at least one of identification, prognosis, and or treatment of a disease in accordance with certain embodiments of the present invention.

### DETAILED DESCRIPTION

Definitions

For the purposes of this specification, unless otherwise indicated, all numbers expressing quantities of ingredients, reaction conditions, and so forth used in the specification are to be understood as being modified in all instances by the term “about.” Accordingly, unless indicated to the contrary, the numerical parameters set forth in the following specification are approximations that can vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g. 1 to 6.1, and ending with a maximum value of 10 or less, e.g., 5.5 to 10. Additionally, any reference referred to as being “incorporated herein” is to be understood as being incorporated in its entirety.

It is further noted that, as used in this specification, the singular forms “a,” “an,” and “the” include plural referents unless expressly and unequivocally limited to one referent. The term “or” is used interchangeably with the term “and/or” unless the context clearly indicates otherwise. Also, the terms “portion” and “fragment” are used interchangeably to refer to parts of a polypeptide, nucleic acid, or other molecular construct.

Unless defined otherwise, all technical and scientific terms used herein have the same meaning as commonly understood by one of ordinary skill in the art. Practitioners are particularly directed to Current Protocols in Molecular Biology (Ansubel) for definitions and terms of the art. Abbreviations for amino acid residues are the standard 3-letter and/or 1-letter codes used in the art to refer to one of the 20 common L-amino acids.

As used herein, “proteomics” is the global analysis of gene expression at the protein level, yielding an overall protein profile for a given cell, tissue, or sample. The comparison of two protein profiles (proteomes) from cells that have been differently treated provides information on the effects the treatment or condition has on protein expression and modification.

“Polypeptide” and “protein” are used interchangeably herein to describe protein molecules that may comprise either partial or full-length proteins. As is known in the art, “proteins”, “peptides,” “polypeptides” and “oligopeptides” are chains of amino acids (typically L-amino acids) whose alpha carbons are linked through peptide bonds formed by a condensation reaction between the carboxyl group of the alpha carbon of one amino acid and the amino group of the alpha carbon of another amino acid. Typically, the amino acids making up a protein are numbered in order, starting at the amino terminal residue and increasing in the direction toward the carboxy terminal residue of the protein.

As used herein, a “protein profile” is at least one protein that is at least partially identified and/or otherwise characterized so that the presence or absence of the protein in any particular sample can be monitored. A protein profile may comprise a single protein or a plurality of proteins. Thus, as used herein, a protein profile may comprise a “fingerprint” that may be used to identify a particular sample or a particular disease or disease subtype. The number of proteins that comprise a protein profile may be at least 50,000, or at least 10,000, or at least 1,000, or at least 100, or at least 20, or at least 10, or at least 5 proteins. Thus, a protein profile may comprise 1-5, or 1-10, or 1-20, or 1-50, or 1-100, or 1-500, or 1-1,000, or 1-5,000, or 1-10,000, or 1-50,000 proteins.

As used herein, the term “marker” refers to a biomolecule, e.g., protein, hormone, prohormone, DNA, RNA (e.g., antisense RNA, small inhibitory RNA) lipid, carbohydrate, and the like, as well as combinations thereof (e.g, a lippoprotein) that is correlated with a particular physiological and/or biochemical process in a subject. For example, a marker may be a protein that is expressed upon the development of an inflammatory response, or a hormone or other biomolecule that is down-regulated due to a particular biochemical signal.

As used herein, the term “upstream” refers to a residue that is N-terminal to a second residue where the molecule is a protein, or 5′ to a second residue where the molecule is a nucleic acid. Also as used herein, the term “downstream” refers to a residue that is C-terminal to a second residue where the molecule is a protein, or 3′ to a second residue where the molecule is a nucleic acid.

A “nucleic acid” is a polynucleotide such as deoxyribonucleic acid (DNA) or ribonucleic acid (RNA). The term is used to include single-stranded nucleic acids, double-stranded nucleic acids, and RNA and DNA made from nucleotide or nucleoside analogues.

The term “vector” refers to a nucleic acid molecule that may be used to transport a second nucleic acid molecule into a cell. The vector may allow for replication of DNA sequences inserted into the vector. The vector may comprise a promoter positioned upstream of the nucleic acid sequence that encodes for a polypeptide of interest to enhance expression of the nucleic acid molecule (i.e., the polynucleotide that encodes for the polypeptide of interest) in at least some host cells. Vectors may replicate autonomously (extrachromasomal) or may be integrated into a host cell chromosome. The vector may be an expression vector capable of producing a protein derived from at least part of a nucleic acid sequence inserted into the vector.

The terms “identity” or “percent identical” refers to sequence identity between two amino acid sequences or between two nucleic acid sequences. Percent identity can be determined by aligning two sequences and refers to the number of identical residues (i.e., amino acid or nucleotide) at positions shared by the compared sequences. Sequence alignment and comparison may be conducted using the algorithms standard in the art (e.g. Smith and Waterman, 1981, Adv. Appl. Math. 2:482; Needleman and Wunsch, 1970, J. Mol. Biol. 48:443; Pearson and Lipman, 1988, Proc. Natl. Acad. Sci., USA, 85:2444) or by computerized versions of these algorithms (Wisconsin Genetics Software Package Release 7.0, Genetics Computer Group, 575 Science Drive, Madison, Wis.) publicly available as BLAST and FASTA. Also, ENTREZ, available through the National Institutes of Health, Bethesda Md., may be used for sequence comparison. In one embodiment, the percent identity of two sequences may be determined using GCG with a gap weight of 1, such that each amino acid gap is weighted as if it were a single amino acid mismatch between the two sequences. Identity may comprise a certain threshold or range. For example, as used herein, the term at least 90% identical thereto includes sequences that range from 90 to 100% identity to the indicated sequences and includes all ranges in between. Similarly the term “at least 70% identical includes sequences that range from 70 to 100% identical, with all ranges in between. The determination of percent identity is determined using the algorithms described here.

As used herein, the term “conserved residues” refers to amino acids that are the same among a plurality of proteins having the same structure and/or function. A region of conserved residues may be important for protein structure or function. Thus, contiguous conserved residues as identified in a three-dimensional protein may be important for protein structure or function. To find conserved residues, or conserved regions of 3-D structure, a comparison of sequences for the same or similar proteins from different species, or of individuals of the same species, may be made.

As used herein, the term “homologue” means a polypeptide having a degree of homology with the wild-type amino acid sequence. Homology comparisons can be conducted by eye, or more usually, with the aid of readily available sequence comparison programs. These commercially available computer programs can calculate percent homology between two or more sequences (e.g. Wilbur, W. J. and Lipman, D. J., 1983, Proc. Natl. Acad. Sci. USA, 80:726-730). For example, homologous sequences may be taken to include an amino acid sequences which in alternate embodiments are at least 75% identical, 85% identical, 90% identical, 95% identical, or 98% identical to each other.

As used herein, the term “interact” refers to a condition of proximity between a first molecule or compound, or portions or fragments thereof, and a second molecule or compound, or portions or fragments thereof. The interaction may be non-covalent, for example, as a result of hydrogen-bonding, van der Waals interactions, or electrostatic or hydrophobic interactions, or it may be covalent.

As used herein, a “ligand” refers to a molecule or compound or entity that interacts with a ligand binding site, including substrates or analogues or parts thereof. As described herein, the term “ligand” may refer to compounds that bind to the protein of interest. A ligand may be an agonist, an antagonist, or a modulator. Or, a ligand may not have a biological effect. Or, a ligand may block the binding of other ligands thereby inhibiting a biological effect.

As used herein, a “modulator compound” refers to a molecule which changes or alters the biological activity of a molecule of interest. A modulator compound may increase or decrease activity, or change the physical or chemical characteristics, or functional or immunological properties, of the molecule of interest. The term “modulator compound” also includes a chemically modified ligand or compound, and includes isomers and racemic forms.

An “agonist” comprises a compound that binds to a receptor to form a complex that elicits a pharmacological response specific to the receptor involved. An “antagonist” comprises a compound that binds to an agonist or to a receptor to form a complex that does not give rise to a substantial pharmacological response and can inhibit the biological response induced by an agonist.

The term “peptide mimetics” refers to structures that serve as substitutes for peptides in interactions between molecules (Morgan et al., 1989, Ann. Reports Med. Chem., 24:243-252). Peptide mimetics may include synthetic structures that may or may not contain amino acids and/or peptide bonds but that retain the structural and functional features of a peptide, or agonist, or antagonist. Peptide mimetics also include peptoids, oligopeptoids (Simon et al., 1972, Proc. Natl. Acad, Sci., USA, 89:9367); and peptide libraries containing peptides of a designed length representing all possible sequences of amino acids corresponding to a peptide, or agonist or antagonist of the invention.

A “subject” may be a human or other mammal. As used herein, subjects may be “patients” who are seeking evaluation of their health, or “controls” who are subjects that are known to exhibit a particular disease (i.e., a positive control), or who, based on medical evaluation as standard in the art, are free of the disease of interest (i.e., a negative control). A patient may exhibit a particular disease, or a patient may be disease-free (as evaluated by a trained physician), but seeking evaluation of his or her overall health.

The term “treating” or “treat” refers to improving a symptom of a disease or disorder and may comprise curing the disorder, substantially preventing the onset of the disorder, or improving the subject's condition. The term “treatment” as used herein, refers to the full spectrum of treatments for a given disorder from which the patient is suffering, including alleviation of one symptom or most of the symptoms resulting from that disorder, a cure for the particular disorder, or prevention of the onset of the disorder.

As used herein, an “effective amount” means the amount of an agent that is effective for producing a desired effect in a subject. The term “therapeutically effective amount” denotes that amount of a drug or pharmaceutical agent that will elicit therapeutic response of an animal or human that is being sought. The actual dose which comprises the effective amount may depend upon the route of administration, the size and health of the subject, the disorder being treated, and the like.

The term “pharmaceutical agent” or “therapeutic agent” includes any agent, such as a protein or polypeptide, that has one or more effects on a biological system as for example, to modify and/or treat a disease of interest.

The term “parenteral” as used herein, includes subcutaneous injections, intravenous, intramuscular, intracisternal injection, or infusion techniques.

The term “pharmaceutically acceptable carrier” as used herein may refer to compounds and compositions that are suitable for use in human or animal subjects. The term “pharmaceutical composition” is used herein to denote a composition that may be administered to a mammalian host, e.g., orally, parenterally, topically, by inhalation spray, intranasally, or rectally, in unit dosage formulations containing conventional non-toxic carriers, diluents, adjuvants, vehicles and the like.

As used herein, an “isolated” biological component (such as a protein) has been substantially separated or purified away from other biological components in the cell of the organism in which the component naturally occurs, i.e., other chromosomal and extrachromosomal DNA and RNA, other proteins and organelles. Also, as used herein the term “purified” does not require absolute purity; rather, it is intended as a relative term. Thus, for example, a purified protein preparation is one in which the protein referred to is more pure than the protein in its natural environment within a cell. The term “separate” or “separation” as used herein denotes spatially dissociation of components, such as biomolecules. The components (for example, proteins or peptides) can be separated based on one or more specific characteristics, such as molecular weight or mass, charge or isoelectric point, conformation, association in a complex, and so forth.

As used herein, a “computer program” comprises a computer-encoded language that encodes the steps required for the computer to perform a specific task or tasks. Also, as used herein, software comprises the computer program(s) used in conjunction with any other operating systems required for computer function.

Also, as used herein, a “computer processor” or “CPU” may include, for example, digital logic processors capable of processing input, executing algorithms, and generating output as necessary in response to the inputs received from the touch-sensitive input device. Such processors may include a microprocessor, such as an ASIC, and state machines. Such processors include, or may be in communication with, media, for example computer-readable media, which stores instructions that, when executed by the processor, cause the processor to perform the steps described herein.

Protein Profiles for Identification and Treatment of Disease

Embodiments of the present invention recognize that one way to understand, and accurately diagnose, different subtypes of diseases, is to describe the genetic processes that contribute to the formation and function of the disease, as well as inflammatory cells and mediators involved. For example, the inflammatory pathways that contribute to chronic rhinosinusitis (CRS) are regulated by multiple genes and genetic processes that contribute to complex genotype and phenotype combinations. The multifactorial nature of this disease has previously made it very difficult to derive a clinically useful classification system. One way of determining the inflammatory profile of a disease is to look at the proteins expressed by genes involved in the pathology of the disease.

Thus, in one embodiment, the present invention comprises a method for identifying a disease in a subject. The method may comprise the step of generating a protein profile comprising at least one protein from a sample isolated from the subject. The method may also comprise the step of comparing the protein profile for the subject to a reference protein profile. In additional embodiments, the method may also comprise the step of correlating the protein profile with a disease of interest.

In an embodiment, the method comprises generating a protein profile comprising at least one protein from a sample isolated from the subject and using the protein profile for patient diagnosis and treatment. For example, in an embodiment, the protein profile may be used for identification of a disease or a disease subtype. Additionally or alternatively, the protein profile may be used for developing a diagnosis for the subject. The protein profile may also be used for determining a prognosis for the subject. In yet another embodiment, the protein profile may be used for following the treatment employed to determine the relative efficacy of a treatment protocol.

In another embodiment, the present invention may comprises a method for identifying a protein that is expressed as a result of a developing a particular disease. The method for identification may comprise the step of isolating samples from a plurality of subjects that exhibit the symptoms of the disease of interest. Also, the method may comprise generating a protein profile for each of the samples. The method may additionally comprise correlating expression of at least one individual protein identified in at least some of the protein profiles to the disease of interest.

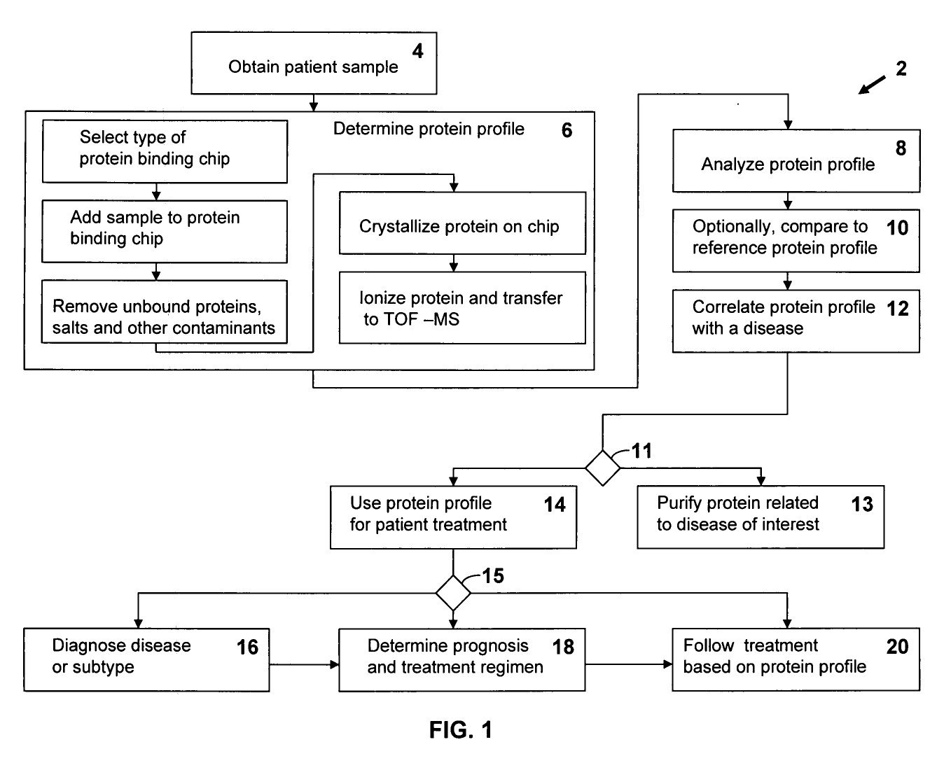

FIG. 1 illustrates an embodiment of a method of the present invention. Thus, in an embodiment, the method may comprise isolating a sample from a subject (4) and generating a protein profile from the sample (6). In an embodiment, the protein profile comprises measurement of at least one specific protein in the sample. The protein that is measured may be a known protein (i.e., the existence of the protein has been previously described) or the protein may comprise a novel protein (i.e., a protein whose existence, sequence and/or function is not known). Alternatively, the protein profile may comprise measurement of a plurality of proteins in the sample. In an embodiment, for the measurement of a plurality of proteins, it is contemplated that some of the proteins may be known, and some of the proteins may be novel. Or, all of the proteins may be known. Or, all of the proteins may be novel.

A variety of methods may be used to generate the protein profile. In certain embodiments, Matrix Assisted Laser Desorption Ionization (MALDI) Mass Spectrometry technology is used. In an embodiment, Surface Enhanced Laser Desorption/Ionization (SELDI) ProteinChip Mass Spectrometry (SELDI) technology may be used as described in more detail herein. However, other techniques known in the art for generating protein profiles may also be used.

The method may include analysis of the protein profile (8). In an embodiment, analysis of the protein profile comprises a statistical analysis and other data manipulation techniques (e.g., signal processing, removal of noise). In an embodiment, techniques for analysis comprise computer statistical and data processing software. For example, analysis of the protein profile may comprise a determination of at least one of the molecular weight (mass), net charge, and or amount of the proteins in the sample.

The method may also comprise the step of comparing the protein profile for the subject's sample to a reference protein profile (10). In an embodiment, the reference profile may be from a healthy control subject who does not exhibit symptoms of the disease of interest (i.e., a negative control). Additionally or alternatively, the reference profile may be from a subject who has a disease of interest (i.e., a positive control). For example, the reference protein profile may include at least one protein having a modified level of expression in a plurality of subjects exhibiting a specific disease of interest. Also, the sample protein profile may be compared to a reference protein profile isolated from the same subject, but at a different point in time (e.g., to monitor progression or remission of the disease). In yet other embodiments, the sample protein profile may be compared to a plurality of a reference protein profiles, as for example, reference profiles generated as diagnostic of a particular disease or disease subtype. In this way, it may be possible to determine whether the sample protein profile matches a particular protein or proteins of interest that are typical of any one disease or disease subtype.

The method may also include the step of correlating the protein profile with the presence or absence of a disease of interest (12). At this point, the user of the method (e.g., a physician) may decide (11) to use the information for the treatment of the subject (14). Alternatively, the data may be used to purify the protein that is related to the disease of interest for further characterization as described in detail herein (13).

If the physician is using the information for treatment of the subject, the physician may decide (15) to use the information in a variety of ways. In an embodiment, analysis of the protein profile, and optionally, comparison of the protein profile to a reference protein profile, provides information about whether the subject has a particular disease or disease subtype. Thus, in an embodiment, the subject's protein profile is used to determine a diagnosis for the subject (16). For example, in an embodiment, the sample protein profile may have a protein in common with a reference protein profile isolated from a subject having a specific disease (or disease subtype) of interest.

The method may be applied to a variety of diseases of interest. In an embodiment, it is applied to the identification of chronic rhinosinusitis (CRS) or a subtype of chronic rhinosinusitis. For example, in alternative embodiments, the method may be used to distinguish at least one of eosinophilic chronic hyperplasic rhinosinusitis (ECHRS), allergic fungal rhinosinusitis, non-eosinophilic rhinosinusitis with polyps or without polyps from each other, any other types of CRS (or combinations thereof), asthma, or other types of inflammatory disorders.

In certain embodiments, the method may be used to develop a prognosis or treatment protocol for the identified disease (18). In an embodiment, the prognosis may comprise a quantification of the severity of the disease or a clinical outcome. For example, the protein profile may indicate that specific markers associated with a particular subtype of a disease are present. Where the markers are proteins that are found in a severe subtype, or are associated with additional pathologies, a more aggressive treatment may be required. If, however, the markers are associated with a less aggressive form of the disease, the protein profile may indicate that the disease is less severe allowing for the use of less aggressive treatment procedures.

In certain embodiments, the method may be used to evaluate a treatment protocol for the identified disease (20). For example, in an embodiment, samples may be obtained from the subject during the course of treatment for the disease of interest. Analysis of the protein profile may indicate that a marker or markers (i.e., a particular protein or proteins) that are specific to the disease are either increasing or decreasing in response to therapy. If the marker is decreasing, it may be inferred that the therapy is effective. If the marker is not decreasing, this can indicate that additional, or more aggressive therapeutic regimens are required. In an embodiment, the protein profile may be used for each of the diagnosis (16), prognosis (18) and to follow the course of treatment (20) as indicated in FIG. 1.

In an embodiment, the protein profile may be correlated with other disease indicators. For example, there may be other disease indicators that can be diagnostic of the disease of interest. Thus, in certain embodiments, the method may comprise correlating the protein profile to a second marker or markers. For example, many diseases are associated with changes in the immune response. Thus, in an embodiment, the protein profile may be correlated with a modification in the amount of at least one cell type that is involved in the immune response. For example, for CRS and subtypes of CRS, the protein profile may be correlated with the extent of sinus mucosa eosinophila. Thus, the protein profile may be correlated with the distribution of activated eosinophils (EG2+) in the sinus mucosa of patients with CRS. In alternate embodiments, the protein profile may also be correlated with the presence of other cell types such as at least one of activated mast cells, fibroblasts or goblet cells.

Or, the protein profile may be correlated with the presence or absence (or changes in relative levels) of non-cellular markers that may be modified in response to the disease. For example, the additional marker correlated to the protein profile may comprise a cytokine, a hormone or prohormone, a lipid or lipoprotein, a receptor or receptor ligand (e.g., agonist, antagonist), a transcription factor, or any other cellular regulatory factor or modulator compound. For example, in an embodiment, the inflammatory agent may comprise a cytokine. For CRS, cytokines that are associated with the disease, such GM-CSF, IL-3, IL-4, IL-5, IL-6, IL-8, IL-9, IL-11, IL-13, Eotaxin-1, or TGF-beta may be measured. In some embodiments, the protein profile may be correlated with dysregulation of cysteinyl leukotrienes and/or sinus mucosa leukotriene levels. In other embodiments, leukotrienes, interleukins, exotoxin, granulocyte-macrophage colony-stimulating factor, tumor necrosis factor alpha, transforming growth factor beta, CD4+ lymphocytes and various cytokines may be measured.

The determination of the protein profile may be made by a variety of methods known in the art, as for example, by two-dimensional (2D) gel electrophoresis (see e.g., 0'Farrel, J. Biol. Chem., 1975, 250: 4007; U.S. Patent Application Publication No.: 2001/0107057). In an embodiment, the method used is a rapid-throughput method to allow for the analysis of multiple samples from patients and/or controls.

The method may be used for the analysis of patient samples. Thus, in alternate embodiments, the method may be adapted to the analysis of biological materials that comprise typical bodily samples such as sputum, saliva, skin cells, blood, serum, urine, hair, nasal and sinus tissue, or the like. The method may, in certain embodiments, allow for the use of such patient samples without any type of pre-purification steps to remove non-protein components.

Alternatively, in an embodiment, the patient samples may be cultured using cell culture methods as known in the art prior to analysis. Proteomic studies can be performed on these sinus mucosa cell cultures to study the effects of disease or toxi substances such as exposure to cigarette smoke, asbestos, or other irritants.

In certain embodiments, the generation of protein profiles may be done using Matrix Assisted Laser Desorption Isonizaton (MALDI) Mass Spectrometry. In an embodiment, Surface Enhanced Laser Desorption/Ionization (SELDI) ProteinChip Mass Spectrometry (SELDI) technology may be used. SELDI can bring to proteomics many of the advances DNA chip technology has brought to genomic analysis and functional genomics. In alternate embodiments, the arrays may be coated with a compound comprising a functional group that is able to capture protein molecules from complex mixtures by the affinity of such proteins for the coating compound (Hutchens et al., Rapid Comm. Mass Spectrom., 1993, 7: 576-580; Merchant et al., 2000, Electrophoresis 21, 1164-1167). In an embodiment, SELDI protein chips may comprise PROTEINCHIP® arrays (Ciphergen Biosystems, Inc.). One advantage of the SELDI technique is that analysis may be performed on blood serum, making in easily applicable in the practice setting.

In an embodiment, SELDI technology may be used to identify the serum protein profiles of patients with different disease subtypes of a disease of interest. In an embodiment, the patients (i.e., subjects) are those having chronic rhinosinusitis, and are undergoing sinus surgery after failure of medical therapy. Thus, the identified signature “fingerprint” protein profiles may be used to improve the diagnosis and/or prognosis of complex diseases such as CRS. Additionally, such signature serum protein profiles may be correlated with the disease's clinical and basic subjective and objective parameters, with the goal of developing a simple blood “fingerprint test” to accurately diagnose the patient's specific disease and/or to evaluate and relevant inflammatory response factors that may play a role in prognosis or therapy.

In an embodiment, a PROTEINCHIP® SELDI chip and suggested protocols may be used. Or, alternative SELDI protein chips may be utilized. In various embodiments, the protein chips may comprise different types of capturing matrices coated onto the surface, such as hydrophobic compounds, ionic compounds, or metal-binding compounds, to optimize protein binding affinities. Additionally or alternatively, protein chips may comprise surfaces that provide covalent immobilization of antibodies, receptors, receptor ligands or other protein binding partners (i.e., compounds that interact with a protein, or a subtype of proteins, of interest), DNA, glycoproteins, or the like, for specific affinity capture in the sample.

In an embodiment, proteins retained on the chip may be analyzed by time of flight-mass spectrometry (TOF-MS). In alternate embodiments, tandem (MS/MS) mass spectrometers may be used. For example, in alternate embodiments, quadrupol-quadrupole, magnetic sector-quadrupole, or quadrupole-time-of-flight MS may be used. With the aid of computerized analysis techniques, a retentate map may be generated depicting the ratio of mass/charge, which in most cases, may correspond to the molecular weight for each of the proteins. The peptide mass profile (peptide fingerprint) obtained from mass spectrometry may be compared with theoretical fragmentation patterns derived from sequence data in genomic databases in order to aid in identifying the proteins. Different spectra may then be combined or compared to elucidate changes of the protein profiles between samples.

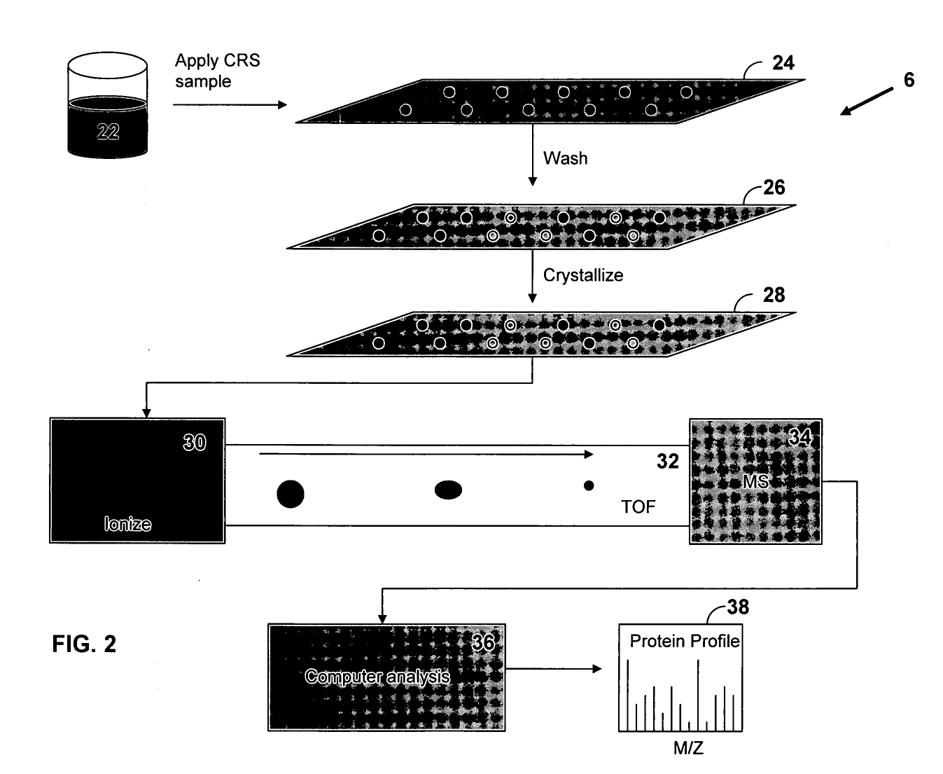

For example, and referring now to FIG. 2, for the method steps of generating of a protein profile (6), a sample (22) may be applied to a chip having either a hydrophobic, anionic, cationic or metal binding surface (24). Next, the chip may be washed, to remove unbound proteins, salts and other contaminants (26). The proteins remaining on the chip surface may then be crystallized, as for example using Sinapinic acid (SPA) or another crystallization reagent (28). Desorption of the crystallized proteins may then be initiated by ionization (30). The released proteins may then be transported through a time of flight (TOF) tube (32) to the mass spectrometer (MS) detector (34) which measures the mass of the protein and using computerized analysis (36) and calculates the charge based on the protein molecule's time of flight. The information is converted to protein mass/charge and includes the amount of each protein (38). The process may be automated and thus, is easily adapted for high throughput analysis.

For detection of peaks, spectra (e.g., in triplicate or duplicate) may be compiled after the completion of the SELDI assay. In an embodiment, mass calibration may be performed using standards. For example, in an embodiment, mass calibration may be performed using the All-in-1 peptide standard spectrum.

The analysis may provide for a subtraction of background noise. The analysis may also provide for a normalization of the peaks relative to an internal standard(s) over a range of molecular weights. For example, for measurement of proteins ranging from 1,000 to 100,000 Da, the peak intensities may normalized using the total ion current from mass/charge of 1000 to 100,000 Da. In an embodiment, detection of peaks may utilize a computer program and software. For example, in an embodiment, Ciphergen Biomarker Wizards software, or similar software, may be used to auto detect protein peaks.

There may be a certain threshold by which signals are designated as individual proteins. In an embodiment, protein peaks may be selected based on a signal to noise ratio. For example, in alternate embodiments, a first pass of signal to noise ratio of at least 10, or at least 5, or at least 3, or at least 2 may be used. Also, various peak threshold values may be used depending upon the level of resolution required. In an embodiment, the minimum peak threshold may be about 0.5%, or about 1%, or about 5%, or about 10%, or about 20%, or about 40% or about 60% of all spectra. In an embodiment, a first pass threshold of about 20% is used.

In an embodiment, there may be more than one cycle of peak selection. The second pass of peak selection may comprise a lower value for the threshold. In an embodiment, the second threshold is about ½, or about ⅕, or about 1/10 or about 1/20 or about 1/50, or 1/100, or 1/500, or 1/1,000, or 1/5,000 of the initial threshold. For example, for a first threshold of about 20%, the second pass of peak selection may be at about 0.2% of the mass window, and the estimated peaks were added.

In an embodiment, the selected protein peaks may then be averaged as clusters. Once the data have been clustered, it may be exported to a data processing system for further analysis. For example, in an embodiment the Ciphergen Biomarker Patterns Software (Wadsworth et al., Arch. Otolaryngol., Head Neck Surg., 2004, 130:98-104) may be used for further classification analysis of the clusters.

A variety of analysis techniques may be used for generating protein profiles. In an embodiment, classification and regression tree analysis (CART) is performed. The analysis may use the common protein peaks identified by SELDI as a means to generate protein profiles (Wadsworth et al., 2004). For example, in an embodiment, CART analysis utilizes a decision classification tree algorithm. The algorithm may be generated based on the identification of protein peaks differentially expressed between the disease samples and the controls. In an embodiment, the classification analysis splits the data into 2 groups or nodes by separating samples by rules based on the presence or absence of a peak sequentially until terminal nodes are reached. In an embodiment, the data is validated using statistical techniques known in the art. For example, classification of data may be performed using ten-fold cross-validation, which uses random numbers to split up the data used for classification tree training for the purpose of testing each classification tree.

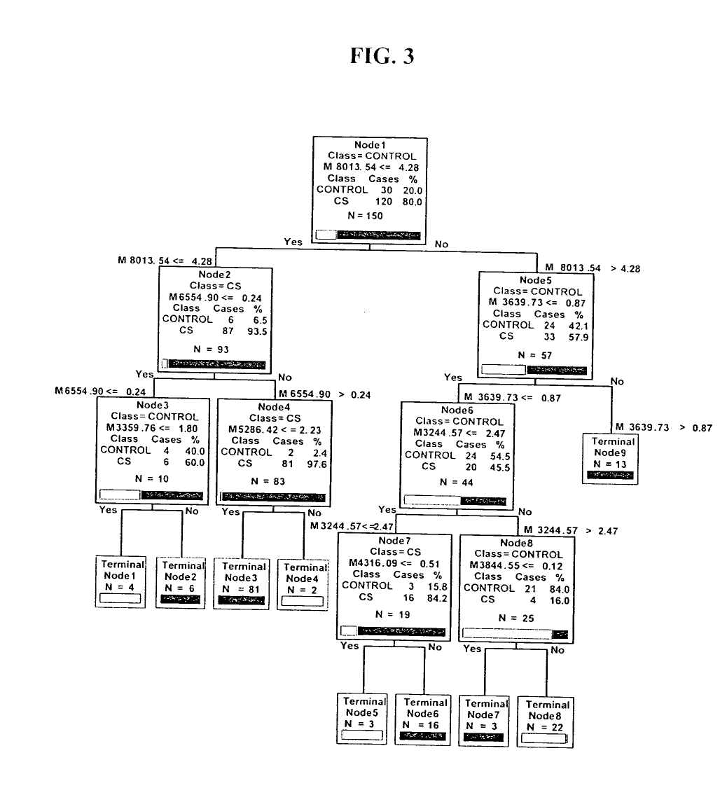

For example, for the analysis of samples from either subjects and/or controls, software (e.g., Ciphergen Biomarker Wizards) may be used for peak detection such that after analyzing a plurality of spectra (e.g., triplicate spectra from both samples and controls), a plurality of clusters generated by the data in the range of 1 kDa to about 130-150 kDa. Thesclusters may then be used in a subsequent classification analysis. In an embodiment, the classification also employs a computer program and software (e.g., Ciphergen Biomarker Pattern Software (BPS)). The analysis may be a tree-structured data analysis (e.g., BPS), which is derived from CART (Classification and Regression Tree), where CART analysis is a nonparametric regression method based on the recursive partitioning method (Grajski et al, IEEE Trans. Biomed., Eng., 1986, 33:1076-86). The classification may then construct a decision tree which correctly classified patients with disease as compared to controls based on a tree having n masses (e.g., peaks) and n+1 nodes (FIG. 3).

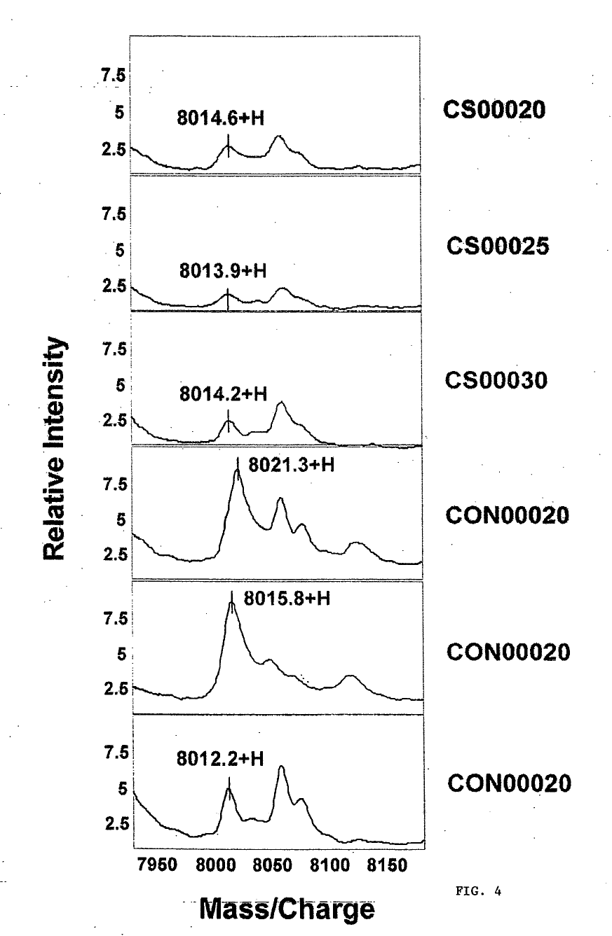

In an embodiment, the classification rule used is straightforward. Thus, if the sample had a peak at a defined mass, with a defined intensity level then the sample may be placed in a particular node and classified as either healthy or having a particular disease. For example, FIG. 3 shows a diagram of Biomarker Pattern Analysis utilized in the classification of chronic sinusitis (CS) (solid) and healthy, negatively screened controls (open) in accordance with an embodiment of the present invention. Each node represents an analytical splitting rule where the samples are split into two daughter nodes. Each node displays the peak mass, the cutoff intensity level, the number of samples and the composition of the samples. Terminal nodes are classified as either Control or CS.

Whereas some of the protein peaks will be specific to the disease, and others may be specific to the controls, many of the peaks may be shared between disease and control. Thus, in an embodiment, analysis with SELDI-TOF-MS generates high-throughput protein profiles that may be analyzed to identify different protein patterns that exist between patients of interest and healthy controls. For example, FIG. 4 shows a peak at about 8,013.54 Da that was upregulated in chronic sinusitis (CRS) patients compared to negative controls (CON). In an embodiment, assay reproducibility may be examined by running a pooled serum quality control sample (QC) in each chip array.

Purification of Proteins Diagnostic of Disease

Once proteins that are specific to the disease of interest are identified, the proteins may be isolated and further characterized. It can be important to know the identity of the protein for the development of future clinical immunoassays and/or to develop methods to produce the protein in large scale amounts for use as a therapeutic. For example, for proteins that are over-expressed in the disease state, antibodies to such proteins may be generated. Such antibodies may be useful therapeutics for the treatment and/or prevention of the disease. Or, for proteins that comprise receptors and/or receptor ligands, antagonists of receptor binding may be developed. Or, for proteins that are under-expressed in the disease state, it may be possible to develop therapeutics that allow for the protein to be provided to the subject. As discussed in more detail herein, such therapeutics may include methods and/or compositions to increase expression of the protein of interest (i.e., the protein having expression levels that are at least in part correlated with the disease of interest) at the genetic level (i.e., by increasing transcription of the gene that encodes the protein or by increasing stability of the protein).

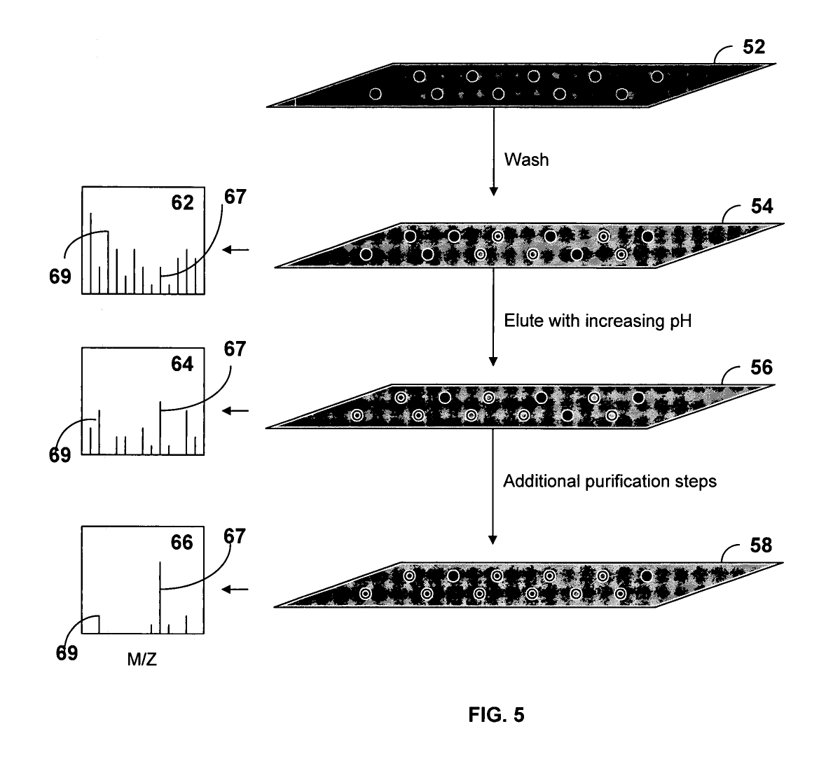

In certain embodiments, the technique used to generate a protein profile may be used to purify a protein of interest. For example, in an embodiment, SELDI may be used to purify proteins that are diagnostic of the disease of interest (FIG. 5). For SELDI-based purification, the sample may be applied to a SELDI chip comprising the appropriate coating (52). The chip may be washed, to remove unbound proteins, salts, and other contaminants (54). By varying chip chemistries and wash stringencies the best strategy for purification of the marker proteins may be established. For example, for purification of blood serum proteins using SELDI, sera may be incubated on an anionic surface, and unwanted proteins removed in an increasingly basic pH wash (56). Proteins retained on the surface may then be analyzed by Time of Flight-Mass Spectrometry (TOF-MS) as described herein.

In an embodiment, other purification methods may be used to purify a protein of interest. Where other purification methods are used, SELDI may be used, in certain embodiments, to monitor the purification. Or, in an embodiment, and again referring to FIG. 5, a combination of SELDI-based purification and other purification methods (58) may be used.

Thus, separation of the protein of interest from the other members of the protein profile may be accomplished by any number of techniques, such as sucrose gradient centrifugation, aqueous or organic partitioning (e.g., two-phase partitioning), non-denaturing gel electrophoresis, isoelectric focusing gel electrophoresis, capillary electrophoresis, isotachyphoresis, mass spectroscopy, chromatography (e.g., HPLC), polyacrylamide gel electrophoresis (PAGE, such as SDS-PAGE), gel permeation, ion-exchange spin columns, and so forth. In these embodiments, SELDI, or other rapid analysis techniques, may be used for monitoring the purification process. Following purification, all potential markers may be characterized by SDS PAGE and mass spectrometry and identified by peptide mapping and/or amino acid sequence analysis.

For example, in an embodiment, the proteins may be separated by size or buoyant density gradient separation method, such as a discontinuous sucrose gradient, that separates the component polypeptides of the sample by the sizes of the complexes in which they participate. Sucrose gradients for the separation of proteins are well known, and may be modified as needed. Such modifications may include the use of a continuous, rather than discontinuous gradient, and different gradient conditions (for instance, different sucrose concentrations or different buffers). The length of the gradient can also be varied, with longer gradients expected to give better overall separation of proteins and protein complexes, and to provide a larger number of fractions that are then each individually analyzed using a denaturing system.

Alternatively or additionally, the individual proteins may be separated by electrophoresis based upon size (e.g., by SDS-PAGE or sizing gel). Other separation techniques may include aqueous two-phase partitioning and non-denaturing agarose gel electrophoresis separation. In other embodiments, separation employs denaturing system such as an isoelectric focusing (IEF) gel, capillary electrophoresis, or isotachyphoresis. Alternatively or additionally, two-dimensional electrophoretic analysis may be used (e.g., Wilkins et al., Proteome Research: New Frontiers in Functional Genomics, Springer-Verlag, Berlin, 1997). As is known in the art, proteins can be visualized on such gels using any of various known stains (e.g., Trypan Blue or SyproRuby™ dye). Also, traditional buffering systems can be used for separating proteins in the component fractionations of the described systems. As is known in the art, the temperature, voltage, and amperage at which individual gels are run also can be modified, as can the speed and duration of gradient equilibration and centrifugation. All such minor variations of conditions that are used to optimize separation conditions are encompassed herein.

Alternatively or additionally, purification of disease-associated proteins may be performed using traditional chromatographic techniques. In an embodiment, high pressure liquid chromatography (HPLC) may be used. Also, a combination of high pressure liquid chromatography (HPLC) and sodium dodecyl sulfate polyacrylamide gel electrophoresis (SDS-PAGE) may be used to purify the protein. The fractions may then be assayed for the protein of interest using SELDI or other methods.

In an embodiment, and again referring to FIG. 5, SELDI retentate maps (62, 64, 66), showing an increase in the purification of a protein of interest 67 from other proteins e.g., 69, or other means of monitoring the protein profile, may indicate increasing contribution of the retained proteins as the purification procedure progresses.

Proteins that are purified or separated (e.g., to generate SELDI rentate maps or other types of protein profiles) may be analyzed and/or identified using any of various techniques known in the art (e.g., Wilkins et al., Proteome Research: New Frontiers in Functional Genomics, Springer-Verlag, Berlin, 1997). Examples of applicable protein identification techniques include protein activity assays, antibody recognition, direct comparison to previous proteomic maps, and database screening for peptide sequence matches.

For example, to identify the protein of interest, the purified protein may be digested with endoproteinases to generate peptides. In an embodiment, such peptides may be analyzed by SELDI. The digestions can, in some cases, be performed directly on the SELDI chip, but can also be done more traditionally in solution from purified protein obtained from HPLC, gel electrophoresis bands, or PVDF transblotted bands, using methods known in the art. Peptides may then be submitted for analysis by SELDI or other techniques, for a determination of molecular masses. The peptides may then be searched against a mass spectrometry database, such as the PROWL database (Rockefeller University) available over the Internet. For example, the programs m/z and PAWS may be used to analyze and interpret peptide fingerprints. If such searches fail to match the protein of interest (i.e., the protein having expression levels correlated with a disease), a partial amino acid sequence of the protein may be determined. Proteins not identified by mass searches may also be characterized using standard techniques for amino acid sequence analysis of peptides, such as separation of peptides by reverse-phase HPLC after endoproteinase digestion, followed by sequencing using a LC-MS/MS.

A variety of database searching and analysis programs may be used for the identification of isolated proteins. In an embodiment, data may collected using Dynamic exclusion, with multiple MS/MS scans for every full MS scan.

In certain embodiments, database searching may be performed. In an embodiment, the database searching may comprise using TurboSEQUEST® to compare the protein/peptide against a human protein database allowing two missed cleavages per peptides. The TurboSEQUEST® software may be used to rapidly compare and correlate acquired MS/MS spectra of peptides, typically generated by enzymatic digestion of proteins, with predicated MS/MS spectra generated from protein or nucleotide sequence database. The results may then be summarized using a scoring algorithm that uses correlation values produced by TurboSEQUEST® to assign statistical significance to the matches obtained. Using this tandem mass spectrometry approach, a complete or partial sequence information may be obtained at the femtomole to picomole level for peptides containing up to 25 amino acid residues. In an embodiment, internal sequence data can often be obtained for peptides up to 30 or 40 residues (Hamilos et al., 1993). Sequences identified may then be searched via the Internet using the BLAST (NLM/I\IIH) and SWISS-PROT programs freely available over the internet.

Systems For the Identification of Protein Profiles For Disease Diagnosis and Treatment

In other embodiments, the present invention also provides systems for determining protein profiles for disease diagnosis and treatment. In an embodiment, the system may comprise a component for generating a protein profile comprising at least one defined protein from a sample isolated from a subject. The system may also comprise a component for comparing the protein profile for the subject to a reference protein profile. In some embodiments, the system may also comprise a component for correlating the protein profile with a disease of interest. The systems of the present invention may be designed for high-throughput analysis of protein profiles.

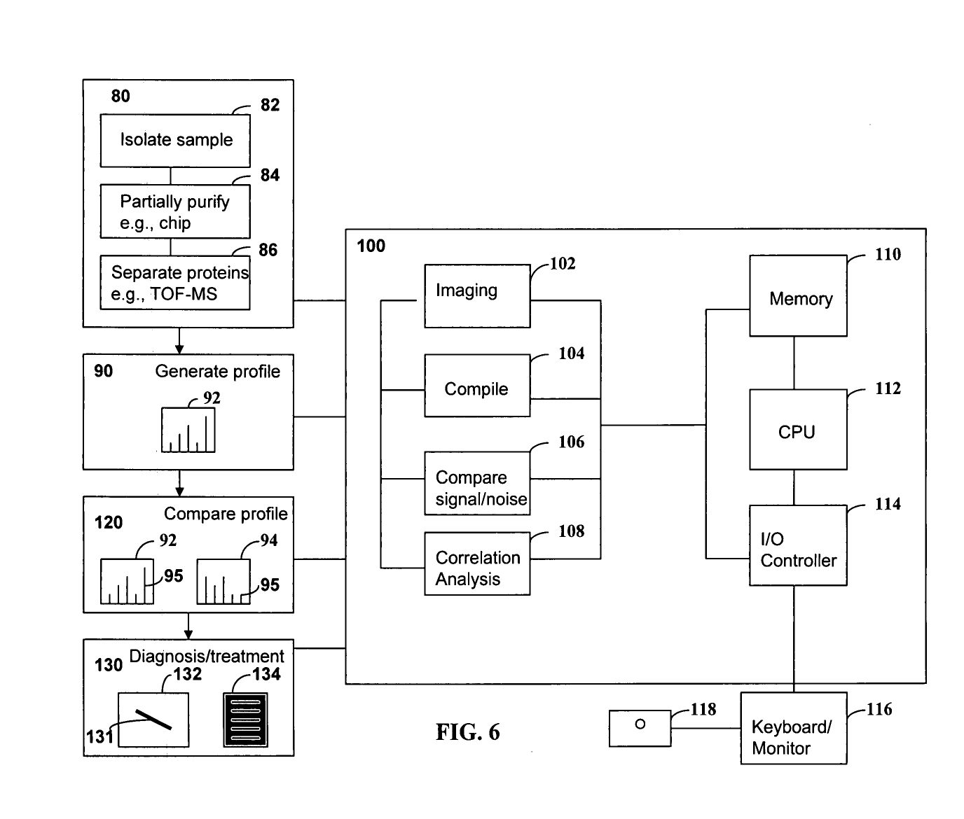

FIG. 6 shows an example embodiment of a system of the present invention. Thus, in an embodiment, the system may comprise a component (80) for preparing the samples to generate a protein profile. In an embodiment, the system may include a component for isolating a sample from a subject (82). The system may also include a component for capturing and isolating the proteins away from other components in the sample (e.g., SELDI chip) (84), and separating the proteins from each other (e.g., TOF-MS) (86).

The system may also comprise a component for generating a protein profile (90). In an embodiment, generation of the protein profile comprises a statistical analysis and other data manipulation techniques (e.g., image processing, removal of noise). In an embodiment, generating the protein profile comprises using computer (100) comprising statistical and data processing software.

For generation of the protein profile, the starting point may comprise a plurality of proteins generated from a MALDI or SELDI chip. The proteins may be separated (86) using TOS-CM as described herein. Alternatively, the data may comprise an electronic image showing the presence or absence of particular proteins bound to an affinity chip. In this case, the system may comprise an imaging system. Thus, in various embodiments, the computer may therefore comprise software for collection of the data (102), e.g., either TOF-MS data or other types of data. The imaging system used may be custom-designed, or may be one of a number of commercially available packages.

Once the data has been collected, it may be compiled (106) and/or transformed if necessary using any standard spreadsheet software (e.g., Microsoft Excel, FoxPro, Lotus, or the like). In an embodiment, the data are entered into the system for each experiment. Alternatively, data from previous runs are stored in the computer memory (110) and used as required.

At each point in the analysis, the user may input instructions via a keyboard (116), floppy disk, remote access (e.g., via the internet) (118), or other access means. The user may enter instructions including options for the run, how reports should be printed out, and the like. Also, at each step in the analysis, the data may be stored in the computer using a storage device common in the art such as disks, drives or memory (110). As is understood in the art, the processor (112) and I/O controller (114) are required for multiple aspects of computer function. Also, in a embodiment, there may be more than one processor.

The data may also be processed to remove noise (104) and to quantify the protein peaks for each protein profile. For example, as described herein, peaks may be selected based on a signal to noise ratio. In some cases, the user, via the keyboard (116), floppy disk, or remote access (118), may want to input variables or constraints for the analysis, as for example, the threshold for determining noise.

In an embodiment, the protein profile comprises measurement of at least one defined protein in the sample. Alternatively, the protein profile may comprise measurement of a plurality of proteins in the sample. A variety of methods may be used to generate the protein profile. In an embodiment, Surface Enhanced Laser Desorption/Ionization (SELDI) technology in combination with time-of-flight (TOF) mass spectrometry (MS) may be used as described in more detail herein. However, other techniques known in the art for generating protein profiles may also be used.

The system may also comprise a component (120) for comparing the protein profile for the subject (92) to a reference protein profile (94). In an embodiment, the reference profile may be from a healthy control subject who does not exhibit symptoms of the disease of interest. Alternatively or additionally, the reference profile may be from a subject who has a disease of interest. For example, in an embodiment, the protein profile(s) include at least one protein (95) having a modified level of expression in a plurality of subjects exhibiting a specific disease of interest. Also, the sample protein profile may be compared to a reference protein profile isolated from the same subject, but at a different point in time (e.g., to monitor progression or remission of the disease). In yet another embodiment, the sample protein profile may be compared to a plurality of a reference protein profiles, as for example, reference profiles generated as diagnostic of a particular disease or disease subtype. In this way, it may be possible to determine whether the sample protein profile matches a particular protein or proteins of interest that are typical of any one disease or disease subtype.

Thus, the system may comprise a component for generating organizing the data into clusters and correlating the presence of a particular peak data with a disease (108). In an embodiment, comparison of the sample protein profile to the reference protein profile uses a computer (100). For example, the computer may comprise software to perform correlation analysis (108). The computer used for correlation of the protein profile to a reference protein profile may be the same or different than the computer used to generate the protein profile.

The system may also comprise a component for correlating expression of a protein (e.g., as a protein profile comprising quantification of a single protein) with the presence or absence of disease of interest (130). For example, the system may comprise a medium in which the analysis is provided in a form, such as a print-out, a report, or an electronic communication (e.g., e-mail or other type of wireless communication) to a physician. In an embodiment, the component for analysis of the protein profile comprises a computer-based system. The computer used for correlating the protein profile with the presence or absence of disease of interest may be the same or different than the computer used to generate the protein profile. For example, the computer may compare the sample protein profile to a reference protein profile to provide an analysis about whether the subject has a particular disease or disease subtype. Thus, in an embodiment, the subject's protein profile is used to determine a diagnosis for the subject which may be provided as a report (134), or added to the patient's chart. For example, in an embodiment, the sample protein profile may have a protein in common with a reference protein profile isolated from a subject having a specific disease (or disease subtype) of interest. Or, the computer analysis may provide a quantitative analysis of a particular protein. In an embodiment, the analysis may provide data (132) regarding the levels of the protein (131) over the course of treatment.

Thus, in an embodiment, the present invention comprises a computer-readable medium on which is encoded programming code for generating protein profiles from samples isolated from a patient. Also in an embodiment, the present invention may comprise a computer-readable medium on which is encoded programming code for analyzing a protein profile. In an embodiment, computer-readable medium may comprise code for comparing the protein profile for the subject to a reference protein profile. Additionally or alternatively, the computer-readable medium may comprise code for using such profiles to generate a prognosis and/or diagnosis for a disease.

Embodiments of computer-readable media include, but are not limited to, an electronic, optical, magnetic, or other storage or transmission device capable of providing a processor with computer-readable instructions. Other examples of suitable media include, but are not limited to, a floppy disk, CD-ROM, magnetic disk, memory chip, ROM, RAM, an ASIC, a configured processor, all optical media, all magnetic tape or other magnetic media, or any other medium from which a computer processor can read instructions. Also, various other forms of computer-readable media may transmit or carry instructions to a computer, including a router, private or public network, or other transmission device or channel, both wired and wireless. The instructions may comprise code from any computer-programming language, including, for example, C, C#, Visual Basic, Visual Foxpro, Java, and JavaScript.

In an embodiment, the system may comprise a computer and programming code embodied on a computer-readable medium. Thus, in an embodiment, the computer-readable medium on which is encoded programming code for analyzing protein profiles in a disease of interest, and correlating such profiles to at least one of diagnosis, prognosis, or treatment.

Articles of Manufacture

Embodiments of the present invention may also comprise articles of manufacture that may be used for the identification of a disease of interest. For example, in an embodiment, the present invention may comprise an article of manufacture comprising a protein profile captured on a medium, wherein the protein profile comprises at least one protein that is correlated with the development and/or propagation of a disease of interest in a subject. In an embodiment, the protein profile comprises a reference profile that may be used for patient diagnosis and/or treatment.

The protein profile may be in a form that can be distributed to physician or other health care professional for use in a clinic and/or private practice. Thus, in an embodiment the medium on which the protein profile is presented comprises at least one of paper, a CD-ROM, a floppy disk, an electronic communication (e.g., an e-mail or other type of wireless communication), a web-site, or a computer hard drive. For example, the article of manufacture may comprise a series of reference protein profiles that are correlated to a particular disease or disease subtype. In an embodiment, the disease is chronic rhinosinusitis or a subtype of chronic rhinosinusitis.

As described herein, it may be important to enable correlation of a protein profile from a subject to other markers that are known to be important in the diagnosis and/or prognosis of the disease. Thus, in an embodiment, the article of manufacture may comprise a reference profile that includes additional markers that are correlated with the development and/or propagation of a disease of interest in a subject. For example, for CRS and subtypes of CRS, the protein profile may be correlated with the extent of sinus mucosa eosinophila. Thus, the article of manufacture comprising a reference protein profile may include data regarding the distribution of activated eosinophils (EG2+) in the sinus mucosa for a particular CRS subtype. In alternate embodiments, the article of manufacture may also include data regarding the presence or absence of other cell types for a particular disease or disease subtype. For example, for CRS, the article of manufacture may also include data regarding the presence or absence of at least one of activated mast cells, fibroblasts or goblet cells. Or, the protein profile may be correlated with the presence or absence (or changes in relative levels) of non-cellular markers that may be modified in response to the disease. For example, the additional marker correlated to the protein profile may comprise a cytokine, a hormone or prohormone, a receptor or receptor ligand, a lipid or lipoprotein, a transcription factor, or any other cellular regulatory factor. In an embodiment, the inflammatory agent may comprise a cytokine. Thus, in embodiment, the article of manufacture may also include data regarding the presence or absence of such cytokines or other biochemical agents. For the diagnosis of CRS, the article of manufacture may also include data regarding the presence or absence of cytokines that are associated with the disease, such GM-CSF, IL-3, IL-4, IL-5, IL-6, IL-8, IL-9, IL-11, IL-13, Eotaxin-1, or TGF-beta may be measured In yet other embodiments, for the diagnosis of CRS, the article of manufacture may also include data regarding the presence or absence of leukotrienes. In other embodiments, data regarding leukotrienes, interleukins, exotoxin, granulocyte-macrophage colony-stimulating factor, tumor necrosis factor alpha, transforming growth factor beta, CD4+ lymphocytes and various cytokines may be included.

Molecular Constructs

In various embodiments, a protein that is identified as being important to the disease of interest may comprise a useful therapeutic agent. Thus, embodiments of the present invention comprise isolated polypeptides/proteins related to a disease of interest, and polynucleotides that may encode for proteins and polypeptides related to a disease of interest. In addition, the present invention comprises cells that produce polypeptides, proteins related to a disease of interest, and polynucleotides that may encode for proteins and polypeptides related to a disease of interest. In an embodiment, the disease is chronic rhinosinusitis or a subtype of chronic rhinosinusitis.

In an embodiment, the protein may be produce by recombinant DNA technology. A recombinant DNA molecule comprising a polynucleotide encoding a polypeptide of interest (e.g., a polypeptide having modified expression in a disease of interest) can be made and expressed by conventional gene expression technology using methods well-known in the art. For example, the practice of the present invention may employ, unless otherwise indicated, conventional techniques of molecular biology, microbiology, recombinant DNA, and immunology, which are within the skill of the art. Such techniques are explained fully in the literature, including Sambrook, et al., Molecular Cloning: A Laboratory Manual 2nd ed. (Cold Spring Harbor Laboratory Press, 1989); DNA Cloning, Vol. I and II, D. N Glover ed. (IRL Press, 1985); B. Perbal, A Practical Guide To Molecular Cloning, Wiley (1984); Gene Transfer Vectors For Mammalian Cells, J. H. Miller and M. P. Calos eds. (Cold Spring Harbor Laboratory, 1987); Methods In Enzymology, Vol. 154 and 155, Wu and Grossman, eds., and Wu, ed., respectively (Academic Press, 1987).

Recombinant constructs comprising a nucleic acid or a polypeptide of interest can be made by well-known recombinant techniques. In this regard, the sequence of interest may be operably linked to one or more regulatory sequences in a suitable vector in a proper reading frame and orientation. The polynucleotide encoding a nucleic acid or polypeptide of interest can be propagated and/or expressed in a prokaryotic or eukaryotic expression vector. In alternate embodiments, a bacterial, mammalian, yeast, or insect cell system may be used for propagation of the recombinant construct.

For example, in an embodiment, the coding sequence of a polypeptide of interest may derived from a complementary DNA (cDNA) made by reverse transcription of cellular RNA from a host cell known to express the gene of interest using methods known in the art. A regulatory sequence comprising a promoter that is operable in the host cell of interest may then be linked upstream (i.e., 5′) of the cDNA sequence using molecular techniques. Other regulatory sequences can also be used, such as one or more of an enhancer sequence, an intron with functional splice donor and acceptance sites, a signal sequence for directing secretion of the recombinant polypeptide, a polyadenylation sequence, other transcription terminator sequences, and a sequence homologous to the host cell genome for insertion of at least a portion of the recombinant polynucleotide into the host genome. Other sequences, such as an origin of replication, can be added to the vector as well to optimize expression of the desired product. Also, a selectable marker may be included in the vector for selection of the presence thereof in the transformed host cells.

The regulatory sequences may be derived from various sources. For example, one or more of them can be normally associated with the coding sequence, or may be derived from, or homologous with, regulatory systems present in the host cell. Alternatively, the promoter may be derived from a gene that is turned on in response to the development of the disease of interest. The various components of the expression vector can be linked together directly or, via linkers that constitute sites of recognition by restriction enzymes as is known in the art.

Any promoter that would allow expression of the nucleic acid that encodes for a polynucleotide or a polypeptide of interest can be used in the present invention. For example, mammalian promoter sequences that can be used herein are those from mammalian viruses that are highly expressed and that have a broad host range.

The promoter may be a promoter that is expressed constitutively in most mammalian cells. Examples of suitable regulatable elements which make possible constitutive expression in eukaryotes are promoters which are recognized by the RNA polymerase III or viral promoters, CMV enhancer, CMV promoter, SV40 promoter or LTR promoters, e.g. from MMTV (mouse mammary tumor virus) and other viral promoter and activator sequences, derived from, for example, HBV, HCV, HSV, HPV, EBV, HTLV or HIV. Examples of regulatable elements which make possible regulatable expression in eukaryotes are the tetracycline operator in combination with a corresponding repressor (Gossen M., et al., 1994, Curr. Opin. Biotechnol., 5, 516-20). Alternatively, expression of the gene of interest genes may take place under the control of tissue-specific promoters. Alternatively, the promoter may be a promoter that is turned on at a particular time in the cell cycle or developmental phase. For example, the constructs may comprise regulatable elements which make possible tissue-specific expression in eukaryotes, such as promoters or activator sequences from promoters or enhancers of those genes which code for proteins which are only expressed in certain cell types. Examples of regulatable elements which make possible cell cycle-specific expression in eukaryotes are promoters of the following genes: cdc25A, cdc25B, cdc25C, cyclin A, cyclin E, cdc2, E2F-1 to E2F-5, B-myb or DHFR (see e.g., U.S. Pat. No. 6,856,185; U.S. Pat. No. 6,903,078; and Zwicker J. and Muller R., 1997, Trends Genet., 13, 3-6).

In another embodiment, an enhancer element can be combined with a promoter sequence. Such enhancers may not only amplify, but also can regulate expression of the gene of interest. Suitable enhancer elements for use in mammalian expression systems are, for example, those derived from viruses that have a broad host range, such as the SV40 early gene enhancer, the enhancer/promoters derived from the LTR of the Rous Sarcoma Virus, and from human cytomegalovirus. Additionally, other suitable enhancers include those that can be incorporated into promoter sequences that will become active only in the presence of an inducer, such as a hormone, a metal ion, or an enzyme substrate, as is known in the art.

In another embodiment of the present invention, a transcription termination sequence may be placed 3′ (i.e., downstream) to the translation stop codon of the coding sequence for the gene of interest. Thus, the terminator sequence, together with the promoter, would flank the coding sequence.

The vector for expression of a polypeptide of interest may also contain an origin of replication such that it can be maintained as a replicon, capable of autonomous replication and stable maintenance in a host. Such an origin of replication includes those that enable an expression vector to be reproduced at a high copy number in the presence of the appropriate proteins within the cell, for example, the 2μ and autonomously replicating sequences that are effective in yeast, and the origin of replication of the SV40 vital T-antigen, that is effective in COS-7 cells. Mammalian replication systems may include those derived from animal viruses that require trans-acting factors to replicate. For example, the replication system of papovaviruses, such as SV40, the polyomavirus that replicate to extremely high copy number in the presence of the appropriate vital T antigen may be used, or those derived from bovine papillomavirus and Epstein-Barr virus may be used. In some cases, the expression vector can have more than one replication system, thus, allowing it to be maintained, for example, in mammalian cells for expression and in a prokaryotic host for cloning and amplification.

In one embodiment, the expression vector can be made to integrate into the host cell genome as an integrating vector. The integrating vector herein may contains at least one polynucleotide sequence that is homologous to the host cell genome that allows the vector to integrate. For example, in one embodiment, bacteriophage insertion sequences or transposon sequences may be used.

In certain embodiments of the present invention, one or more selectable markers can be included in the expression vector to allow for the selection of the host cells that have been transformed. Selectable markers that can be expressed in a host cell include genes that can render the host cell resistant to drugs such as tunicamycin, G418, ampicillin, chloramphenicol, erythromycin, kanamycin (neomycin), and tetracycline. Selectable markers also include biosynthetic genes, such as those in the histidine, tryptophan, and leucine biosynthetic pathways, such as ade2, his4, leu2, trp1, or that provide the host cells with the ability to grow in the presence of toxic compounds, such as a metal, may be used.

Compositions

In alternate embodiments, the polypeptide or protein that is identified using the methods or systems of the present invention may comprise therapeutic potential. Thus, the protein/polypeptide may, in certain embodiments, be mixed with a pharmaceutically acceptable carrier to produce a pharmaceutical composition that can be administered for the treatment of a cell having a cosmetic function.

In various embodiments, the protein/polypeptide used in the compositions of the present invention may include analogs and/or fragments that retain the biological activity of the full-length polypeptide. Such analogs may include post-translationally modified polypeptides, for example, peptide mimetics, analogues generated by glycosylation, acetylation, or phosphorylation of the polypeptide. Analogs can be also made by conventional techniques of amino acid substitution, deletion, or addition, as for example, by site-directed mutagenesis. In one embodiment, a fragment of the polypeptide of interest can be made by deleting residues from the nucleic acid that encodes for the polypeptide as is known in the art. In another embodiment, polypeptide of interest can be expressed as a fusion protein by linking, in the correct frame and orientation, the coding sequence of the polypeptide to the coding sequence of another molecule. The fusion protein may be designed to increase the stability or the correct processing of the polypeptide. Or, the polypeptide can be conjugated to other molecules suitable for its intended use. For example, the polypeptide can be conjugated to a binding partner (e.g., a ligand) to a receptor that is recognized by the cell of interest.

The compositions of the present invention may be prepared as a formulation that is suitable for parenteral administration (including subcutaneous, intramuscular and intravenous administration), topical administration (including nasal or ophthalmic administration), oral administration, or rectal administration, as well as other routes of administration or combinations thereof.

The compositions of the present invention may conveniently be presented in a dosage form and may be prepared by any of the methods well known in the art of pharmacy. The formulations may include bringing the active compound into association with a carrier which constitutes one or more accessory ingredients. In general, the formulations are prepared by uniformly and intimately bringing the active compound into association with a liquid carrier, a finely divided solid carrier, or both, and then, if necessary, shaping the product into desired formulations.

In an embodiment, the composition may comprise a formulation for parenteral administration. Formulations suitable for parenteral administration may conveniently comprise a sterile aqueous preparation of the active compound, which is preferably isotonic with the blood of the recipient.

In an embodiment, the compositions of the present invention may comprise a topical formulation. Topical formulations can be comprised of either dissolving or suspending the compositions in a media such as mineral oil, petroleum, polyhyrodxy alcohols or other bases used for topical pharmaceutical formulations. The addition of other ingredients, such as cocoa butter or aloe may be desirable. Depending upon the intended use of the preparation, other components can be incorporated into it to prepare a preparation having desired rheological properties.

The topical formulation may, in certain embodiments, comprise a nasal spray. For example, nasal spray formulations may comprise purified aqueous solutions of the active compound with preservative agents and isotonic agents. Such formulations are preferably adjusted to a pH and isotonic state compatible with the nasal mucous membranes.

Alternatively, the topical formulation may comprise an opthalmic formulation. Ophthalmic formulations may be prepared by a similar method to the nasal spray, except that the pH and isotonic factors are preferably adjusted to match that of the eye.

In an embodiment, the composition may comprise an oral formulation. Formulations of the present invention suitable for oral administration may be presented as discrete units such as capsules, cachets, tablets or lozenges, each containing a predetermined amount of a potentiating agent as a powder or granules; as liposomes; or as a suspension in an aqueous liquor or non-aqueous liquid such as a syrup, an elixir, an emulsion or a draught. For example, a tablet may be made by compression or molding, optionally with one or more accessory ingredients. Compressed tablets may be prepared by compressing in a suitable machine, with the active compound being in a free-flowing form such as a powder or granules which is optionally mixed with a binder, disintegrant, lubricant, inert diluent, surface active agent or dispersing agent. Molded tablets comprised of a mixture of the powdered active compound with a suitable carrier may be made by molding in a suitable machine. Or, a syrup may be made by adding the active compound to a concentrated aqueous solution of a sugar, for example sucrose to which may also be added any accessory ingredient(s). Such accessory ingredient(s) may include flavorings, suitable preservatives, an agent to retard crystallization of the sugar, and an agent to increase the solubility of any other ingredient, such as a polyhydric alcohol, for example glycerol or sorbitol.

For each of the compositions of the present invention, the formulations may include one or more accessory ingredient(s) selected from diluents, buffers, flavoring agents, binders, disintegrants, surface active agents, thickeners, lubricants, preservatives (including antioxidants) and the like, as is known in the art.

### EXAMPLES

### Example 1

### Protein Profiles in CRS

Cysteinyl leukotrienes can promote eosinophil-mediated inflammation, mucous gland secretion, proliferation of mesenchymal tissue, and may contribute to remodeling and fibrosis. Prior work indicated that CysLT may elevated in patients with asthma compared to patients without asthma (Arango et al., 2002). In addition, chronic hyperplastic rhinosinusitis with nasal polyposis (CHRS/NP) may be associated with excessive expression of CysLT12 (Kountakis et al., 2004). In some cases, clinical and basic science parameters may be combined to recommend a severity classification system for the severity of CRS (Kountakis et al., 2004). The inventors have had experience analyzing the different inflammatory and clinical profiles seen in patients with different subtypes of CRS such as CHRS/NP. Such analyses indicated that numerous inflammatory profiles may exist in patients with CRS. Since inflammatory mediators may be ineffective in resolving the disease, the different inflammatory profiles seen in patients may therefore be influenced by the genetic make up of these patients.

Studies were undertaken to evaluate whether patients with different inflammatory profiles display different signature “fingerprint” serum protein profiles. For these experiments, serum samples were prospectively collected. Ninety-six CRS patients requiring Functional Endoscopic Sinus Surgery (FESS) and thirty-eight normal controls (negative by the Rhinosinusitis Task Force guidelines, nasal endoscopy, and sinus CT) were included. Samples were analyzed by SELDI-TOF-MS as described herein. Data was analyzed using a Ciphergen Biomarker Wisards and Biomarker Pattern Software to process the spectral data and classify disease status.

It was found that SELDI generated protein profiles ranging from about 0 to 100 kiloDaltons (kDa). Classification tree analysis correctly identified patients with CRS with 77.08% sensitivity and 65.79% specificity and a positive predictive value of 88.1%. Sensitivity is defined as the proportion of subjects with sinusitis who have a positive proteomics corroboration with the clinical symptoms. Specificity is the proportion of subjects without sinusitis who have a negative proteomics test for sinusitis. Underexpression of a 8.3 kDa protein was seen in 92.5% CRS patients.

### Example 2

### Patient Samples

A. Enrollment Criteria

Polyp and sinus tissue were obtained from 95 subjects referred for CRS after informed consent. Study subjects were selected based on medical history of chronic rhinosinusitis that will be confirmed by computer tomography (CT) scan and nasal endoscopy. A careful history and physical examination was performed and all patients were questioned about their past medical history and medication usage. The diagnosis of CRS was made according to the criteria established by the Rhinosinusitis Task Force of the American Academy of Otolaryngology-Head and Neck Surgery (Lanza et al., 1997). All patients were treated with the maximum medical therapy for CRS (Lund, V. J., Otollaryngol., Clin, North Am., 38:1301-10, 2005) for at least 4 weeks and if they remained symptomatic, and a CT of the sinuses with 3 mm coronal sections was obtained. Patients returned for follow-up within 1-2 hours after the CT was performed, at which time the decision for surgery was made based on CT findings, endoscopic examination and persistence of CRS symptoms. Individuals to be studied were selected from the patient group recommended for Functional Endoscopic Sinus Surgery (FESS) after failure of appropriate medical management with persistent symptoms.

The diagnosis of nasal polyposis was made based on nasal endoscopy and surgical findings. The diagnosis of allergic fungal rhinosinusitis (AFRS) was made according to the established criteria by Bent and Kuhn (Bent et al., Otolaryngol Head Neck Surg., 1994, 111:580-588). These criteria include: (1) Type I hypersensitivity by history, skin test, or serology; (2) nasal polyposis; (3) characteristic radiographic findings; and (4) eosinophilic mucus demonstrating fungus without tissue invasion. The diagnosis of CHRS/NP is made according to the criteria by Steinke et al (1997), including: (1) nasal polyposis; (2) activated eosinophils (EG2+) in polyp tissue; and (3) no evidence of allergic mucin.

B. SELDI Control Group

Control subjects (n=38) participated in the study as per an informed consent approved protocol. A history and information about sinus symptoms was obtained. Subjects were selected for the control group if they did not satisfy any of the diagnostic criteria for rhinosinusitis established by the Rhinosinusitis Task Force of the American Academy of Otolaryngology-Head and Neck Surgery (Lanza et al., 1997). Subjects were considered as candidates for the control group if they did not have any history of sinusitis for at least 6 months and had a negative sinus CT. None of the subjects in the control group had any other inflammatory diseases such as rheumatoid arthritis, inflammatory bowel disease or autoimmune disease as was determined by history and the absence of use of anti-inflammatory medications. Thus, both study patients and controls were screened for history of any rheumatologic or autoimmune or any inflammatory condition other than chronic sinusitis that would contribute to serum inflammatory protein expression. Any subjects who had a history of these diseases or taking medications treating these conditions were excluded from the study. Healthy control volunteers who met the criteria for diagnosis of CRS based on the Rhinosinusitis Task Force/SAHP guidelines were also excluded from the study. All patients gave informed consent prior to participation.

C. Specimen collection.

At the time of surgery, blood was drawn for analysis and sinus mucosal/polyp tissue was sent for pathologic examination. Polyp tissue was collected and frozen (at −80° C.) for further analysis. Serum samples were centrifuged, and the serum was aliquoted into 500 microliter (μL) aliquots and frozen at −80° C. until SELDI analysis. A quality control serum was used to assure the sample processing and SELDI instrument were in adequate performance.

### Example 3

### Proteomics

A. Use Of SELDI To Generate A Protein Profile

For the initial proteomic experiments, 40 CRS and 10 control samples were used. Additional samples were added in subsequent studies. Serum samples were processed robotically using Biomek 1000 to increase the degree of reproducibility. The IMAC-3 copper treated chip array (Ciphergen Biosystems, Inc., Fremont, Calif.) was used for SELDI analysis as previously described (Adam et al., Cancer Res., 2002, 62:3609-3614) with modification. Triplicate runs were performed for each serum sample with the random placement of each sample in a 96 well bioprocessor format. Briefly, serum samples were prepared for SELDI analysis by vortexing 20 μL of serum with 30 μL of 8M urea with 1% 3-[(3-cholamidopropyl) dimethylammonio]-1-propanesulfonic acid (CHAP) in phosphate buffered saline (PBS) at 4° C. for 10 minutes. 100 μL of IM urea with 0.125% CHAP was added to the serum mixture and vortexed briefly; PBS was added to make a 1:5 dilution of the serum mixture which was then added to the ProteinChip array. After 30 minutes incubation at room temperature, the protein chips were washed with PBS and air-dried. One microliter (μl) of saturated sinapinic acid solution in 0.5% TFA and 50% acetonitrile was applied to each array twice, allowing the array to dry between each application. The SELDI instrument (Ciphergen Protein Biology System IIc, Ciphergen Biosystems, Inc.) was used with an autoloader, which increases the high-throughput significantly. The protein chips were assayed with a laser intensity of 180 and a sensitivity of 8. A total of 192 shots were collected and averaged for each sample. The All-in-1 peptide molecular mass standard (Ciphergen Biosystems, Inc.) was used to generate a peptide standard spectrum for mass accuracy calibration.

B. SELDI-TOF-MS Analysis

All triplicate spectra were compiled after the completion of the SELDI assay. Mass calibration was performed using the All-in-1 peptide standard spectrum. The default background subtraction was applied and the peak intensities were normalized using the total ion current from mass/charge of 1000 to 100,000 Da. The Ciphergen Biomarker Wizards was used to auto detect protein peaks. Protein peaks were selected based on a first pass of signal to noise ratio of 3 and a minimum peak threshold of 20% of all spectra. This process was completed with a second pass of peak selection at 0.2% of the mass window, and the estimated peaks were added. These selected protein peaks were averaged as clusters and exported to the Ciphergen Biomarker Patterns Software (Wadsworth et al., Arch. Otolaryngol., Head Neck Surg., 2004, 130:98-104) for further classification analysis.

C. Classification and Regression Tree Analysis (CART)

Classification and regression tree analysis (CART) was performed using the common protein peaks identified by SELDI as previously described (Wadsworth et al., 2004). A decision classification tree algorithm was generated based on the identification of protein peaks differentially expressed between CRS and negatively-screened control samples. The classification analysis splits the data into 2 groups or nodes by separating samples by rules based on the presence or absence of a peak sequentially until terminal nodes are reached. Classification of test data was performed using ten-fold cross-validation, which uses random numbers to split up the data used for classification tree training for the purpose of testing each classification tree.

D. Results

i. Detection of CRS

Ciphergen Biomarker Wizards software was used for peak detection in this study. After analyzing 263 spectra, it resolved 180 clusters in the range of 1 kDa to 133 kDa. These clusters were used in the subsequent classification analysis. The Ciphergen Biomarker Pattern Software (BPS) was employed for the classification analysis. BPS is a tool developed by Ciphergen for tree-structured data analysis, which is derived from CART (Classification and Regression Tree). CART analysis is a nonparametric regression method based on the recursive partitioning method (Grajski et al, IEEE Trans. Biomed., Eng., 1986, 33:1076-86). The classification analysis (for the study of 40 patients and 10 controls) constructed a decision tree which correctly classified patients with CRS with 89.2% sensitivity and 76.7% specificity. This classification tree used 8 masses (8013.54 Da, 6554.90 Da, 3639.73 Da, 3359.76 Da, 5286.42 Da, 3244.57 Da, 4316.09 Da, 3844.55 Da) to construct a tree with 9 terminal nodes.

The classification rule used was straightforward and is illustrated in FIG. 3 which shows an analysis to classify samples as chronic rhinosinusitis (CS) or controls (CON). If the sample had a peak at 3639.73 Da with intensity >0.87 and a peak at intensity higher than 0.15, then the sample was placed in terminal node 9 and classified as CRS (i.e., CS) (FIG. 3) (solid bars). A sample placed in node 8 has a peak at 3844.55 Da and was designated as a normal control if it has an intensity >0.12, whereas an intensity of ≦0.12 identifies it as CRS, and so forth. Five out of eight peaks used in the classification tree of CRS and healthy negatively screened controls had significantly different intensity levels between these 40 CRS and 10 control samples. The remaining three peaks did not have significantly different intensity levels between the whole study cohort, but they served to give significant discriminate power in the subset of samples. Analysis with SELDI-TOF-MS generates high-throughput protein profiles that may be analyzed to identify different protein patterns that exist between patients of interest and healthy controls. For example, FIG. 4 shows a peak at about 8,013.54 Da that was upregulated in CRS patients compared to negative controls.

The sample size precluded separation of training and blinded test sets. The whole sample set was used as a training set and then a 10 fold validation was performed on samples as a test set. This 10-fold validation test correctly classified CRS patients with 89.17% sensitivity, 76.67% specificity and a positive predictive value of 93.86%.

ii. Reproducibility of SELDI Assay

The assay reproducibility was examined by running a pooled serum quality control sample (QC) in each chip array. A total of 63 chip arrays were used for this study that contained 63 QC spectra. There were 271 clusters detected and the coefficient of variance (CV) was calculated for mass and intensity. An average CV of 0.06% and 27% were observed for mass and intensity, respectively.

E. Purification of Sinusitis-Associated Proteins

Initial protein purification can be attempted directly on the chip array since it is expected that most of the sinusitis-associated proteins will be in low abundance. For example, initial experiments indicated that an 8.3 kDa protein was underexpressed in CRS samples. Thus, the 8.3 kDa protein from control subjects may be purified to determine its identity.

For SELDI-based purification, chip chemistries and wash stringencies are varied to determine the best strategy for purification of the marker proteins. For example, purification of serum proteins using SELDI, serum proteins may be incubated on an anionic surface, and unwanted proteins removed in an increasing pH wash. Proteins retained on the surface may then be analyzed by TOF-MS as described. The retentate maps will indicate increasing contribution of the retained proteins as wash conditions are varied.

If the use of SELDI for initial purification of sinusitis-associated proteins is unsuccessful, traditional chromatographic techniques (utilizing gel permeation and ion-exchange spin columns, and gel electrophoresis) can be used. Thus, purification of the 8.3 kDa protein underexpressed in subjects having CRS was partially purified using HPLC and SDS-PAGE. SELDI may still be used for monitoring the purification process. Following purification, all potential markers may be characterized by SDS PAGE and mass spectrometry and identified by peptide mapping and/or amino acid sequence analysis as described herein.

F. Peptide mass fingerprinting and amino acid sequence analysis

The SELDI system can be used to obtain peptide fingerprints for the selected biomarkers. Purified protein can be digested with endoproteinases to generate peptides, which are then analyzed by SELDI. The digestions can, in some cases, be performed directly on the SELDI chip, but can also be done more traditionally in solution from purified protein obtained from HPLC, gel electrophoresis bands or PVDF transblotted bands. Methods for digestion from gel slices and PVDF blotted material are known in the art. Peptides can then be submitted to SELDI analysis, which can provide molecular masses (SELDI calibrated with internal MW standards for accuracy) that can be searched against mass spectrometry databases, such as the PROWL database at Rockefeller University from the Internet. The programs m/z and PAWS can be used to analyze and interpret the peptide fingerprinting. If such searches fail to match with a known protein, a partial amino acid sequence of the protein may be determined. Any proteins not identified by mass searches can be further characterized by amino acid sequence analysis of peptides separated by reverse-phase HPLC after endoproteinase digestion, by sequencing using a LC-MS/MS. Data can be collected using Dynamic exclusion, with three MS/MS scans for every full MS scan.

Database searching may be performed using TurboSEQUEST® against a human protein database allowing two missed cleavages per peptides. This software is used to rapidly compare and correlate acquired MS/MS spectra of peptides, typically generated by enzymatic digestion of proteins, with predicated MS/MS spectra generated from protein or nucleotide sequence database. The results can be summarized using a newly devised scoring algorithm that uses correlation values produced by TurboSEQUEST® to assign statistical significance to the matches obtained. Using this tandem mass spectrometry approach, complete or partial sequence information may be obtained at the femtomole to picomole level for peptides containing up to 25 amino acid residues. In favorable cases some internal sequence data can often be obtained for peptides up to 30 or 40 residues (Hamilos et al., 1993). Sequences identified may be searched via the Internet using the BLAST (NLM/I\IIH) and SWISS-PROT programs freely available over the internet.

### Example 4

### Detection of Related Contributory Factors

A Sinus Mucosal/Polyp Eosinophils

At the time of surgery, sinus mucosal/polyp tissue was removed and sent for pathologic examination. For this type of analysis, the inflammatory infiltrate is examined and the preponderance of eosinophils is evaluated in a blinded fashion. All specimens are examined microscopically and the number of eosinophils are counted per high-power field (HPF, 40×, 0.238 mm²). Counts are performed for 5 separate high-power fields per specimen. These five counts are averaged to calculate the average number of mucosal/polyp eosinophils per HPF.

B. Distribution Of Activated Eosinophils (EG2+) In The Sinus Tissue

To measure activated eosinophils, tissue is stained for EG2, which is a marker for activated eosinophils. The tissue may be fixed in 4% paraformaldehyde for paraffin embedding and then the paraffin blocks stained with anti-human EG2 at a standardized (e.g., 1:150) dilution (Pharmacia). Immunostaining may be performed using a standard Vectastain Avidin-Biotin complex kit (Vector Laboratories). Eosinophils are considered activated if they stained for EG2 (EG2+). The distribution of activated eosinophils in sinus tissue may then be correlated with proteomics results.

C. Sinus Tissue CysLT

The importance of the dysregulation of CysLTs in CRS may be examined and levels of sinus tissue CysLT correlated with serum protein profiles. As, systemic steroids can dramatically influence tissue inflammation and leukotriene production, patients who use oral steroids within a two-week period prior to surgery are excluded from the study. In addition, no steroids will be administered intraoperatively.

Sinus mucosal/polyp tissue may be obtained for leukotriene C4 quantification as previously described (Steinke et al., 2003). The tissue is homogenized in ethanol to extract the lipid fraction, so as to stabilize the leukotrienes and to remove cellular proteins. The precipitate is then centrifuged, the supernatant dried and resuspended in 80% ethanol, and loaded on a C-18 reverse-phase cartridge (Sep-Pak, J&W Scientific, Folsom, Calif.). The C-18 cartridge is rinsed with ultra-pure water followed by high-pressure liquid chromatography grade hexane, and the cysteinyl leukotrienes subsequently eluted with ethanol:water (90:10).

The eluted cysteinyl leukotrienes can be quantified using a high-sensitivity competitive (sandwich) enzyme immunoassay according to the manufacturer's directions (Cayman Chemical, Ann Arbor Mich.). The data may be analyzed by comparison to known cysteinyl leukotriene standards. This enzyme immunoassay has a sensitivity to 5-10 pg, and detects leukotriene C4, leukotriene D4, and to a lesser extent leukotriene E4. Data are expressed as picograms (pg) of cysteinyl leukotriene C4 per gram of sinus tissue in the original homogenate.

### Example 5

### Statistical Considerations

Statistical analyses will be performed using the JMP-IN software (version 4.0.4). The Pearson product moment correlation will be used to perform regression analysis. The student t-test will be used to compare means between variables and the chi-squared test was used to compare variables reported as frequencies. Standard errors of the means will be calculated using the Means/Anova command of the JMP-IN software. Statistical significance is achieved when p<0.05.

All patents, publications and abstracts cited above are incorporated herein by reference in their entirety. The foregoing is considered as illustrative only of the principal of the invention. Since numerous modifications and changes will readily occur to those skilled in the art, it is not intended to limit the invention to the exact embodiments shown and described, and all suitable modifications and equivalents falling within the scope of the appended claims are deemed within the present inventive concept. All patents, patent publications, and references are incorporated by reference in their entireties herein.

That which is claimed is:

1. A method for identifying a disease in a subject comprising:
(a) generating a protein profile comprising at least one defined protein from a sample isolated from the subject;
(b) comparing the protein profile for the subject to a reference protein profile; and
(c) correlating the protein profile with a disease of interest.

2. The method of claim 1, wherein the disease of interest is chronic rhinosinusitis or a subtype of chronic rhinosinusitis

3. The method of claim 2, wherein step (b) comprises comparing the protein profile from the sample with a reference protein profile from a second subject having a known disease.

4. The method of claim 1, further comprising an additional step (d) of developing at least one of a diagnosis, prognosis or treatment protocol for the identified disease.

5. The method of claim 4, wherein the prognosis comprises a quantification of the severity of the disease or a clinical outcome.

6. The method of claim 1, further comprising correlating the protein profile to at least one additional marker.

7. The method of claim 6, wherein the additional marker comprises a modification in the amount of at least one cell type.

8. The method of claim 7, wherein the modified cell type comprises at least one of eosinophils, activated lymphocytes, activated mast cells, fibroblasts, or goblet cells.

9. The method of claim 6, wherein the additional marker comprises an anti-inflammatory agent.

10. The method of claim 9, wherein the anti-inflammatory agent comprises at least one of GM-CSF, IL-3, IL-4 or IL-5.

11. A method for identifying proteins that are expressed as a result of a developing a disease of interest comprising:
(a) isolating a plurality of samples from a plurality of subjects that exhibit the symptoms of the disease of interest;
(b) analyzing a protein profile obtained from each of the samples; and
(c) correlating expression of at least one individual protein identified in at least some of the protein profiles to the disease of interest.

12. The method of claim 11, wherein the disease of interest is chronic rhinosinusitis or a subtype of chronic rhinosinusitis.

13. An isolated protein identified by the method of claim 11.

14. An isolated polynucleotide encoding the protein of claim 13.

15. An isolated cell comprising a recombinant nucleic acid molecule comprising the polynucleotide of claim 14.

16. A system for determining a diagnosis and/or prognosis of a specific disease in a subject comprising:
(a) a component for generating a protein profile comprising at least one defined protein from a sample isolated from a subject;
(b) a component for comparing the protein profile for the subject to a reference protein profile; and
(c) a component for correlating the protein profile with a disease of interest.

17. An article of manufacture comprising a protein profile captured on a medium, wherein the protein profile comprises at least one protein that is correlated with the development and/or propagation of a disease of interest in a subject.

18. An article of manufacture of claim 17, wherein the medium comprises at least one of paper, a CD-ROM, a floppy disk, a web-site, or a computer hard drive.

19. The article of manufacture of claim 18, further comprising a profile for additional markers that are correlated with the development and/or propagation of a disease of interest in a subject.
